(19) United States
(12) Patent Application Publication
(10) Pub. No.: US 2026/0047868 A1
(43) Pub. Date: Feb. 19, 2026

(54) External Fixation System

(71) Applicant: Tyber Medical LLC, Bethlehem, PA (US)

(72) Inventors: Robert Flavin, Enniskerry (IE); Jeffrey Tyber, Breinigsville, PA (US); David Hannah, Nazareth, PA (US)

(73) Assignee: Tyber Medical LLC, Bethlehem, PA (US)

(21) Appl. No.: 19/103,643

(22) PCT Filed: Aug. 30, 2023
(86) PCT No.: PCT/US2023/031462

§ 371 Date: Feb. 13, 2025

Related U.S. Application Data
(60) Provisional application No. 63/402,963, filed on Sep. 1, 2022.

(51) Int. Cl. A61B 17/66; A61B 17/00

(52) U.S. Cl. CPC A61B 17/66; A61B 2017/00398; A61B 2017/00407

(57) ABSTRACT

An external fixation system includes a first elongate rod having a first ratchet end and a first fixation end; and a second elongate rod having a second ratchet end and a second fixation end. A ratchet assembly is attached to the first ratchet end and the second ratchet end. The ratchet assembly is configured to compress the first and second elongate rods to each other. A first fixation member is attached to the first fixation end and a second fixation member is attached to the second fixation end.

### BACKGROUND OF THE INVENTION

### Field of the Invention

The invention relates to an external fixation system that can be used to compress or distract adjacent bone portions.

### Description of the Related Art

External fixation can be used as a primary means of stabilizing a realigned fracture, as a means of providing sustained distraction or portable traction, to neutralize a relatively tenuous internal fixation, or to immobilize a limb segment. Advantages of external fixation include avoidance of additional operative dissection, potential facilitation of wound care, and no need for a second operation to remove implants. External fixation can be also used in the situation of infected cases where internal fixation is contra-indicated and also vascular compromised patients.

Disadvantages of external fixation, on the other hand, include putting anatomic structures at risk, possibility of infection of the sites where the pins and wires enter the body, causing joint contractures, and extending the time of bone healing. Further, distraction osteogenesis, which uses external fixation, is the process of bone lengthening which is highly reliant on patient compliance.

Besides repairing bone fractures, external fixators are also used to lengthen limbs in people with short limbs. In a majority of cases, it is used for lengthening legs, wherein the femur, or the thigh bone, is cut in a surgical procedure and external fixators are positioned on either side of the fracture to push the two ends of the bone apart in a gradual procedure. The gap gets filled by bone, which grows into the vacant space.

It would be beneficial to provide an external fixation system that can aid in temporary fixation and alternatively provide compression or distraction of adjacent bone portions.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is an external fixation system that includes a first elongate rod having a first ratchet end and a first fixation end and a second elongate rod having a second ratchet end and a second fixation end. A ratchet assembly is attached to the first ratchet end and the second ratchet end. The ratchet assembly is configured to compress or distracted the first and second elongate rods to each other. A first fixation member is attached to the first fixation end and a second fixation member is attached to the second fixation end.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view of an external fixation system according to an exemplary embodiment of the present invention;

FIG. 2 is a reverse perspective view of the external fixation system of FIG. 1;

FIG. 3 is a sectional view of the ratchet assembly used in the external fixation system of FIG. 1;

FIG. 4 is a perspective view of an alternative embodiment of a ratchet system for use in the external fixation system of FIG. 1;

FIG. 5 is a side elevational view of a screw used with the system of FIG. 1;

FIG. 6 is a side elevational view of an exemplary rod support used with the system of FIG. 1;

FIG. 7 is a side elevational view of an exemplary octarod used with the system of FIG. 1;

FIG. 8 is a top plan view of an alternative exemplary octarod used with the system of FIG. 1; and

FIG. 9 is a perspective view of an external fixation assembly using a plurality of the systems of FIG. 1 and octarods of FIG. 7.

### DETAILED DESCRIPTION

U.S. Provisional Patent Application Ser. No. 63/402,961, entitled “Ligament/Tendon Suture with Paired Anchor System”, invented by this inventor (Attorney Docket No. TYB-056); U.S. Provisional Patent Application Ser. No. 63/402,962, entitled “Screw Capture System for Calcaneal Fracture”, invented by this inventor (Attorney Docket No. TYB-057); U.S. Provisional Patent Application Ser. No. 63/402,965, entitled “Screw Blade Capture System”, invented by this inventor (Attorney Docket No. TYB-058); U.S. Provisional Patent Application Ser. No. 63/402,967, entitled “Repair Plate System from Inside/Out”, invented by this inventor (Attorney Docket No. TYB-059); U.S. Provisional Patent Application Ser. No. 63/402,964, entitled “Talar Repair Jig”, invented by this inventor (Attorney Docket No. TYB-060); U.S. Provisional Patent Application Ser. No. 63/402,971, entitled “Syndesmosis Repair Jig Assembly and Method of Use invented by this inventor (Attorney Docket No. TYB-061); U.S. Provisional Patent Application Ser. No. 63/402,969, entitled “Distal Tibial Osteotomy System”, invented by this inventor (Attorney Docket No. TYB-062); and U.S. Provisional Patent Application Ser. No. 63/402,970 entitled “Achilles Tendon Repair Jig invented by this inventor (Attorney Docket No. TYB-064), are all incorporated herein by reference in their entireties.

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or”. That is, unless specified otherwise, or clear from context, “X employs A or B”is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

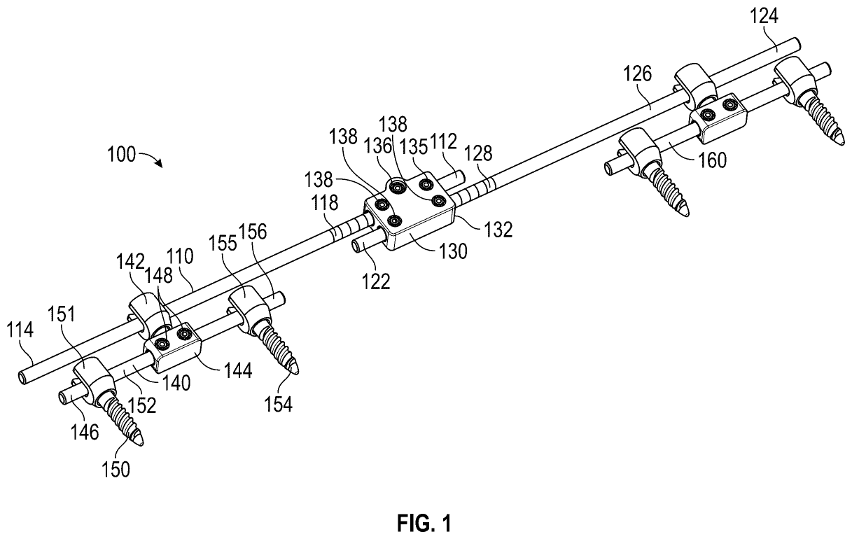

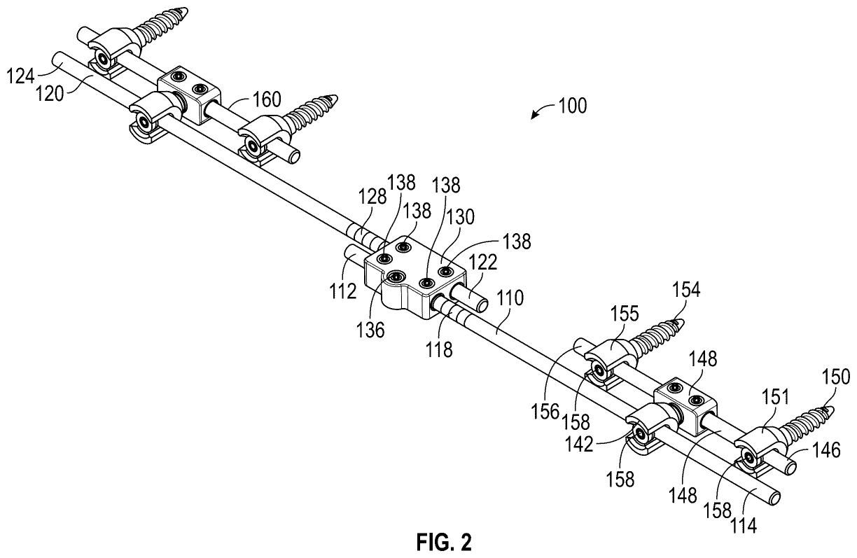

Referring to FIGS. 1-2, an external fixation system 100 (“system 100”) according to a first exemplary embodiment of the present invention is shown. System 100 can be used to compress adjacent separate bones or bone portions together, such as, for example, after a fracture, to assist in temporary fixation. Alternatively, system 100 can be used to distract adjacent bones away from each other, such as for distraction arthroplasty indications, including, but not limited to, pilon fractures, talar fractures, comminuted proximal periarticular knee fractures, and distal radius fractures. System 100 also be used for distraction osteogenesis either through manual turning or motorized computer program turning.

System 100 includes a first elongate rod 110 having a first ratchet end 112 and a first fixation end 114. First ratchet end 114 includes a first tooth rack 116.

System 100 also includes a second elongate rod 120 having a second ratchet end 122 and a second fixation end 124. Second ratchet end 124 includes a second tooth rack 126, facing first tooth rack 116.

A ratchet assembly 130 attached to the ratchet end 112 and second ratchet end 122 and is configured to alternately compress the first and second elongate rods 110, 120 to each other or distract first and second rods 110, 120 away from each other, depending on the direction operation of ratchet assembly 130. Ratchet assembly 130 includes a housing 132 that houses a pinion 134 engages with first rack 116 and second rack 126.

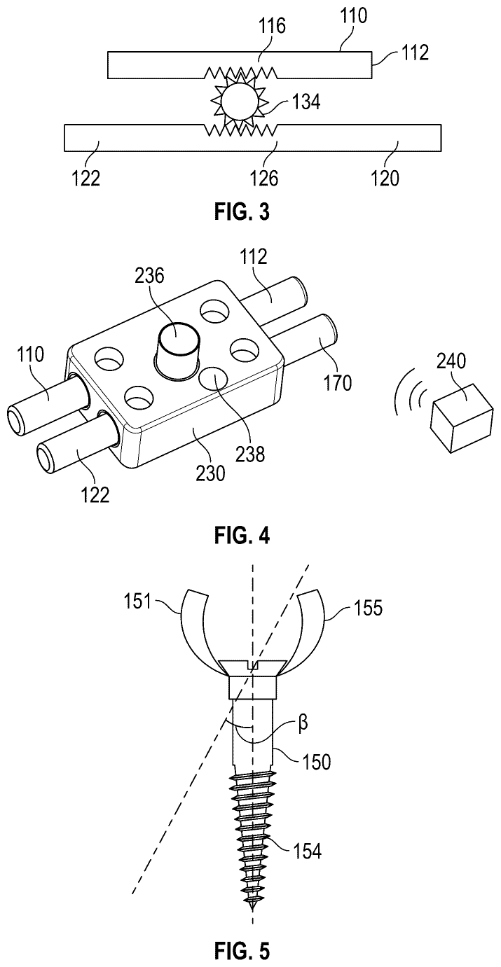

In an exemplary embodiment, as shown in FIG. 3, ratchet assembly 130 can be manually operated, with a driver receiver 136 extending through housing and engaged with pinion 134 such that, when a driver (not shown) is inserted into driver receiver 136 and rotated, driver receiver 136 rotates pinon 134 to slide racks 116, 126 in desired opposing directions to either compress or distract rods 110, 120.

In an alternative embodiment, shown in FIG. 4, instead of a manual rotation, a motor 236 can be provided such that ratchet assembly 230 is motor operated. In this embodiment, ratchet assembly 230 can include electronic receiver 238 configured to electronically receive a signal to operate motor 236. Further, a transmitter 240 is provided and configured to remotely transmit a signal to receiver 238 to operate motor 236.

Transmitter 240 can be any type of electronic device, such as a mobile phone, an electronic pad, or other electronic device that can display the amount of compression or distraction to a third party. Transmitter 240 can transmit an amount or distance that elongate rods 110, 120 are to be compressed or distracted, with that amount or distance picked up by receiver 238, which in turn operates motor 236 to rotate and compress or distract elongate rods 110, 120 the desired amount or distance.

Optionally, referring back to FIGS. 1 and 2, rods 110, 120 can also include indicia 118, 128 that can be used to indicate the amount of compression or distraction that is provided by system 100. Alternatively, an electronic readout can be provided based on the amount of movement of rods 110, 120 relative to ratchet assembly 130.

In either the manual or the motorized embodiment of ratchet assembly 130, 230, screws 138 in ratchet assembly 130, located above one of elongate rods 110, 120, can be tightened down onto their respective elongate rods 110, 120 to secure elongate rods 110, 120 in place relative to ratchet assembly 130.

Assembly 100 further includes a first fixation member 140 attached to the first fixation end 114 and a second fixation member 160 attached to the second fixation end 124. First and second fixation members 140, 160 can be identical, so only first fixation member 140 will be described in detail.

First fixation member 140 comprises a polyaxial head 142 rotatably attached to a rod support 144 and a fixation rod 146 extending through rod support 144. Fixation rod 146 is longitudinally translatable along rod support 144 and, when fixation rod 146 is translated a desired distance, screws 148, located above fixation rod 146, can be tightened down onto fixation rod 146 to secure fixation rod 146 in place relative to rod support 144.

A first polyaxial screw 150 is attached to rod 146 on a first side 152 of rod support 144 and a second polyaxial screw 154 is attached to rod 146 on a second side 156 of rod support 144. First and second screws 150, 156 are each longitudinally translatable along fixation rod 146. As shown in FIG. 5, screws 150, 154 can pivot to an angle β of up to about 45 degrees relative to its heads 151, 155, respectively. Set screws 158 can be used to secure heads 142, 151, 155 to their respective rods 110, 146.

The assembly that makes the polyaxial screws 150, 151 and 154, 155 can move in multiple conical angles until a rod 146 and rod support 144 is assembled. Once screws 150, 154 are tightened, the angle of polyaxial screws 150, 154 is fixed. This allows for the fixation points to be selected first and the external rod 146 to be assembled second.

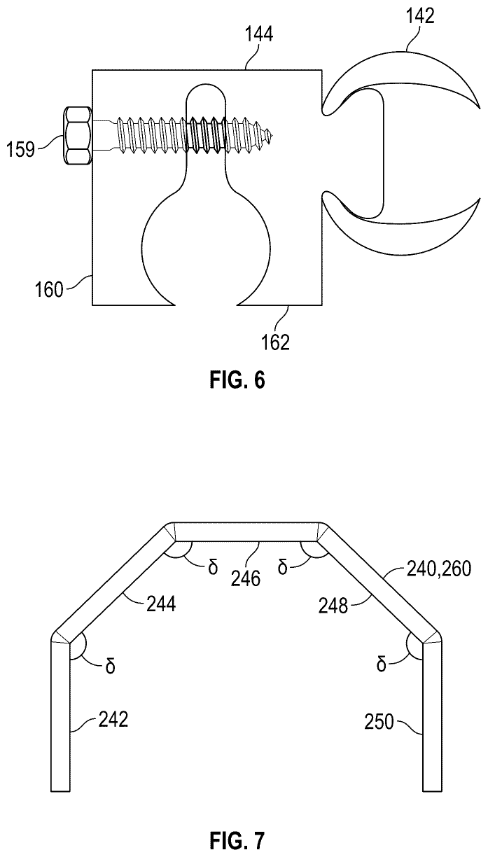

Referring to FIG. 6, rod support 144 can include a static compression portion with screw 159 that can be tightened or loosened to compress or distend opposing jaws 160, 162 to secure or release rod 146 along a single axis, as well as a dynamic portion that includes head 142 that can rotate about the axis.

Referring to FIG. 7, instead of fixation members 140, 160 being a straight rod 146, rods that form the general shape of half of an octagon, with five sides of an octagon, called octarods 240, 260 can be used as the fixation members. Octarods 240, 260 can be attached at their free ends to form an octagon. Alternatively, octarods can be complete octagonal shapes, such as octagon 280, shown in FIG. 9.

As described above with respect to fixation members 140, 160, octarods 240, 260 are also identical to each other, so only octarod 240 will be described. Each octarod includes five segments 242, 244, 246, 248, 250 that are connected to adjacent segments at an angle δ of about 135 degrees, although those skilled in the art will recognize that the angles can be different.

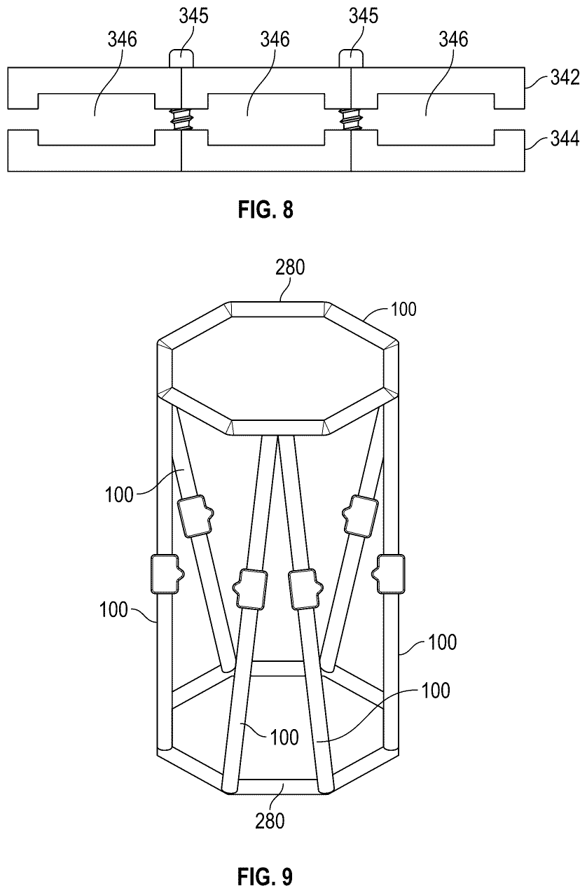

Octarod 240 can be single pieces, as shown in FIG. 7. Alternatively, an octarod 340 can be multipiece, constructed from mirror image halves 342, 344 across a central plane. When halves 342, 344 are assembled together using screws 345, as shown in FIG. 8, passages 346 are formed. Passages 346 are sized to allow screws, such as screws 150, 154 to be installed therethrough or installed prior to the octarod halves 342, 344, with the octarod halves 342, 344 secured around screws 150, 154.

As shown in FIG. 9, a pair of superior octarods 240, 260 and a pair of inferior octarods 240, 260 can be connected by a plurality of systems 100, forming a polyaxial frame system, so that the superior octarods 240, 260 and inferior octarods 240, 260 can be compressed or distracted. Note that the polyaxial screws 150, 154 and polyaxial frame system described above remain polyaxial until a locking nut (or bolt) locks down, thereby making the polyaxially movement mono-axial.

Further, while an octagonal shape of an external fixation system is shown, those skilled in the art will recognize that the external fixation system can have other shapes, such as hexagonal or any other appropriate shape.

Further, the rods disclosed above can have differing diameters, including but not limited to, 6 mm, 8 mm, and 10 mm. Additionally, the screws described above can have variable nonthreaded shaft lengths. Also, in an exemplary embodiment, a gap spanned by free ends of octarod 180 can be between about 160 mm and about 220 mm.

It will be further understood that various changes in the details, materials, and arrangements of the parts which have been described and illustrated in order to explain the nature of this invention may be made by those skilled in the art without departing from the scope of the invention as expressed in the following claims.

We claim:

1. An external fixation system comprising:
a first elongate rod having a first ratchet end and a first fixation end a second elongate rod having a second ratchet end and a second fixation end;
a ratchet assembly attached to the first ratchet end and the second ratchet end, the ratchet assembly configured to compress the first and second elongate rods to each other;
a first fixation member attached to the first fixation end; and
a second fixation member attached to the second fixation end.

2. The external fixation system according to claim 1, wherein the ratchet assembly is configured to alternatively distract the first and second elongate rods from each other.

3. The external fixation system according to claim 1, wherein the first ratchet end comprises a first tooth rack, the second ratchet end comprises a second tooth rack, and the ratchet assembly comprises a pinion engage with the first rack and the second rack.

4. The external fixation system according to claim 1, wherein the ratchet assembly is manually operated.

5. The external fixation system according to claim 1, wherein the ratchet assembly is motor operated.

6. The external fixation system according to claim 5, wherein the ratchet assembly further comprises an electronic receiver configured to electronically receive a signal to operate the motor.

7. The external fixation system according to claim 6, further comprising a transmitter configured to remotely transmit a signal to the receiver t operate the motor.

8. The external fixation system according to claim 1, wherein the first fixation member comprises a head rotatably attached to a rod support.

9. The external fixation system according to claim 8, wherein the first fixation member further comprises a fixation rod extending through the rod support.

10. The external fixation system according to claim 9, wherein the fixation rod is longitudinally translatable along the rod support.

11. The external fixation system according to claim 9, further comprising a first screw attached to the rod on a first side of the rod support.

12. The external fixation system according to claim 1, further comprising a second screw attached to the rod on a second side of the rod support.

13. The external fixation system according to claim 11, wherein the first screw is longitudinally translatable along the fixation rod.

14. The external fixation system according to claim 1, wherein the first fixation member comprises a straight rod.

15. The external fixation system according to claim 1, wherein the first fixation member comprises a rod having five segments.

16. The external fixation system according to claim 15, wherein the second fixation member comprises five segments such that the first and second fixation members can be attached to each other to form an octagon.
